(19) United States
(12) Patent Application Publication
(10) Pub. No.: US 2015/0297749 A1
(43) Pub. Date: Oct. 22, 2015

(54) LOW-DENSITY LIPOPROTEIN ANALOGUE NANOPARTICLES, AND COMPOSITION COMPRISING SAME FOR TARGETED DIAGNOSIS AND TREATMENT OF LIVER

(71) Applicant: POSTECH ACADEMY-INDUSTRY FOUNDATION, Pohang-si, Gyeongsangbuk-do (KR)

(72) Inventors: Sei Kwang HAHN, Pohang (KR); Won Ho KONG, Pohang (KR); Kitae PARK, Jeonju (KR)

(21) Appl. No.: 14/430,945

(22) PCT Filed: Oct. 2, 2013
(86) PCT No.: PCT/KR2013/008834

§ 371 Date: Mar. 25, 2015

(30) Foreign Application Priority Data
Oct. 11, 2012 (KR) 10-2012-0113188

(51) Int. Cl. A61K 47/48; A61K 51/04; C12N 15/113; A61K 51/12

(52) U.S. Cl. CPC A61K 47/48907; A61K 47/48215; A61K 47/48053; A61K 51/1251; A61K 51/0493; C12N 15/1136; A61K 47/48123; C12N 2320/30; C12N 2310/14

(57) ABSTRACT

This disclosure relates to a low density lipoprotein-like cationic solid lipid nanoparticle targeting liver cells including parenchyma cells and non-parenchyma cells, a composition for liver target delivery, a composition for diagnosis and/or treatment of liver disease comprising the same, and a method for liver targeting of an active ingredient.

### FIELD OF THE INVENTION

The present invention relates to low density lipoprotein-like cationic solid lipid nanoparticles targeting liver cells including parenchyma cells and non-parenchyma cells, and a composition for liver-targeted delivery, a composition for diagnosis and/or treatment of liver disease comprising the same.

### BACKGROUND OF THE INVENTION

Liver is involved in metabolism of carbohydrate, protein, fat, hormone and the like, and dexoxification and sterilization and the like, and performs the essential functions for maintaining homeostasis. During these processes, liver is continuously exposed to various external antigens including bacteria, viruses, and toxic substances. The continuous and repeated exposure to antigens causes continuous and serious damage to liver tissue, and inflammation and injury induced during the recovery of the damage may lead to various liver diseases.

Most acute and chronic liver diseases due to environmental or genetic factors cause complicated imbalance or abnormality of parenchymal cells (i.e., hepatocyte) and non-parenchymal cells (i.e., Kuffer cell, sinusoidal endothelial cell, hepatic stellate cell) constituting liver, and the abnormality may lead to necrosis, apoptosis, or proliferation, and alteration of cell function, and the like.

The imbalance between parencymal cells and non-parencymal cells is frequently involved in would healing and scar formation processes such as fibrogenesis, fibrosis, fibrolysis of extracellular matrix, and the like, and these processes may lead to a fibrogenic process. Although the fibrogenic process is complicated and dynamic, it may be characterized by ECM deposition due to increased production of extracellular matrix (ECM) resulting from the simultaneous reactions of various cells or cell types as mentioned above, and the excessive production of ECM may be finally developed to liver fibrosis and liver cirrhosis.

Recently, a fibrotic process is considered to be dynamic and simultaneously reversible, and in this regard, most effective anti-fibrotic therapy is to inhibit or treat the progression of underlying diseases. For example, if HBV or HCV virus is removed from hepatitis B virus (HBV or hepatitis C virus (HCV) infected patient, reversal of fibrosis resulting from the virus infection may be led. However, unfortunately, acute or chronic liver disease treatment technologies or treatment strategies up to date have inadequate or limited therapeutic effects for many underlying diseases including hepatotropic virus infection. In practice, undesirable biodistribution and the resulting side effects and the like of the formulations comprising nucleoside analogues such as Lamivudine and the like, currently clinically used for treatment of acute and chronic hepatotropic virus infection are continuously pointed out as problems, and thus, there are many restrictions on development of novel drugs.

Thus, currently, there is an urgent need for development of specific and appropriate therapeutic technology including acute or chronic intractable liver disease therapy and antifibrotic therapy that can inhibit or treat the resulting liver fibrosis and cirrhosis.

For this reason, for the past tens of years, liver has received attention as a target organ of various gene therapies, but although various therapies including specific antifibrotic therapy for acute and chronic liver diseases and the involved hepatic fibrosis and cirrhosis were tested, they were not successful.

Up to recently, there have been a lot of understandings and progressions in acute and chronic liver diseases including virus infections and hepatic fibrosis and cirrhosis through the studies on cell-based and molecule-based therapies. Particularly, since it has been proved that various effector cell groups perform important functions in a fibrogenic process, hepatic stellate cell (HSC), which is conventional effector cell, is being recognized as most important fibrogenic cell in liver. It is known that HSC passes through complicated activation processes by scarring, and activated HSC produces a quantity of ECM with profibrotic cytokine and growth factor to induce deposition of fibrous tissue or scar, thereby rendering the fibrotic process persistent. Thus, fibrogenic cell types including HSC are receiving attention as target cells in the treatment of fibrotic disorder.

And, recently, it was found that connective tissue growth factor, CTGF) expressed by effector cells such as HSC is a highly fibrogenic molecule and performs a critical role in hepatic fibrosis occurrence, and thus, CTGF knockdown using siRNA (small interfering RNA) and the like is receiving attention as an effective antifibrotic therapy.

Even recently, cellular distribution and transcription of CTGF in the fibrogenic process are in doubt and considered to be influenced by the cause of a disease or passage of time, but for liver, broad cell types including the above mentioned activated HSC and hepatocyte including periportal and pericentral fibroblast and myoblast, bone marrow derived cell, fibroblast derived epithelial cell, biliary epithelial cell and endothelial cell are receiving attention as an important source of CTGF and an effector cell.

Meanwhile, although antiviral nucleoside analogues widely used for treatment of acute and chronic liver diseases resulting from hepatotropic virus infection including HBV and HCV provides comparatively effective inhibition of virus replication, there have been obstacles to clinical development of various antiviral nucleoside analogues due to undesirable biodistribution of so far developed therapeutic drugs such as Lamivudine and the resulting side effects. Recently, nucleic acid-based therapy mediated by siRNA and the like is receiving attention as novel therapeutic strategy since it has been found out that hepatotropic virus replication and expression can be effectively inhibited thereby.

However, there is a difficulty in the development due to the absence of delivery system that can liver-specifically and effectively deliver therapeutic drugs, nucleic acids and the like effective for treatment of liver diseases resulting from acute and chronic infections and hepatic fibrosis and cirrhosis resulting from various acute and chronic liver diseases. Thus, there is an urgent need for development of liver-specific highly efficient delivery system for therapeutic drugs and nucleic acids that can effectively remove disease processes causing fibrosis and antifibrotic therapy, and can be widely applied for treatment of various liver diseases.

Meanwhile, lipoprotein including low density lipoprotein (LDL) is a spherical macromolecule, and consists of a lipophilic core, an emulsified shell surrounding the core, and one kind or many kinds of apolipoprotein (apo). Particularly, apolipoprotein is directly involved in receptor mediated binding and endocytosis inducement in the processes of stabilization of lipoprotein, recruitment of lipid, control of enzyme activation and particularly metabolism. Since natural low density lipoprotein is involved in lipid delivery through blood, is biodegradable due to the endogenous nature, does not induce an immune response, and does not recognized by reticuloendothelial system, it has received attention as effective and specific delivery system for delivering drugs and nucleic acids in the cells. However, the endogenous nature became a cause for limiting large scale pharmaceutical application thereof, and thus, recombinant lipoproteins using commercially available or synthesizable lipids and recombinant apolipoproteins and the like have been prepared and studied by various study groups.

Particularly, when low density lipoprotein-like nanoparticles are injected into blood, serum apoliprotein is adsorbed on the surface of the nanoparticles due to high blood affinity and strong hydrophobic interaction with low density lipoprotein-like nanoparticles, thus very similarly mimicking the metabolism of natural liproprotein that is uptaken in the liver cell by liver specific recognition of the apolipoprotein adsorbed on the surface of the nanoparticles and various receptors such as liproprotein receptor (LDLr), chylomicron remnant receptor and the like abundant in the liver cell and receptor-mediated endocytosis.

Simultaneously, it was confirmed that by reconstructing the outer shell of low density lipoprotein-like nanoparticle by introducing cationic lipid DC-cholesterol and fusogenic lipid DOPE, electrostatic interaction with a phospholipid bilayer constituting cell membrane may be increased and high destabilization to cell membrane may be achieved, thus enabling highly efficient intracellular delivery of nanoparticles and a construct comprising the same.

And, recently, it was found out that endogenous low density lipoprotein can induce activation of HSC in the processes of activation of HSC, most important effector cell for hepatic fibrosis, and epithelial-mesenchymal transition through the activation, and thus, it is expected that various antifibrotic therapies based on the competition between reconstructed low density lipoprotein-like nanoparticles and the endogenous LDL receive attention.

### SUMMARY OF THE INVENTION

### Technical Problem

As described above, therapy using drugs for treatment of liver diseases comprising nucleosides and nucleoside analogues such as Zeffix and Hepsera currently widely used for treatment of hepatitis, and gene therapy through nucleic acid delivery have problems of undesirable biodistribution of therapeutic drugs or formulations, high immunogenicity and a lack of specificity, which should be solved for the ultimate purpose of more effective and efficient treatment.

Due to the limitations of the existing therapies, for development of therapies of intractable liver diseases including acute and chronic liver diseases and the resulting hepatic fibrosis and cirrhosis, development of highly efficient, low toxic and cell-specific drug or nucleic acid delivery technology should precede.

However, undesirable biodistribution of drug or nucleic acid to be delivered, high immunogenicity and deficiency in specificity and the like are pointed out as the limitations, and thus, there is an urgent need for development of effective therapeutic delivery technology.

Thus, the present invention is created to overcome problems and limitations of the conventional therapies for acute and chronic liver disease and hepatic fibrosis and cirrhosis including HBV and HVC infections. The inventors confirmed that low density lipoprotein-like nanoparticles with a predetermined composition can mediate delivery of therapeutic drugs and nucleic acids and the like liver-specifically and with high efficiency through metabolic behavior mimicking natural low density lipoprotein (LDL). Moreover, by incorporating a diagnostic composition as well as a therapeutic composition in the low density lipoprotein-like nanoparticle, a composition having functions of diagnosis and treatment of liver disease was completed.

Thus, it is an object of the invention to provide low density lipoprotein-like (LDL-like) nanoparticles reconstructed and surface modified by mimicking lipid constituents of naturally existing LDL, and use of the nanoparticles for diagnosis and treatment of acute and chronic intractable liver diseases.

More specifically, one aspect of the invention provides a LDL-like nanoparticle reconstructed by mimicking the constituents of natural LDL, consisting of a core-shell structure comprising a solid lipid core containing cholesteryl ester and triglyceride; and an outer shell containing cholesterol, fusogenic lipids, cationic lipids, and lipid-PEG (polyethylene glycol) polymer.

Another aspect provides a composition for delivery of model drugs and/or therapeutic nucleic acids, and a composition for diagnosis and treatment simultaneously having non-invasive imaging diagnosis function, comprising the LDL-like nanoparticles as an active ingredient.

### Technical Solution

The present invention relates to treatment and liver imaging diagnosis technologies mediated by low density lipoprotein-like nanoparticles for delivering therapeutics highly efficiently and specifically for the whole liver cells including parenchyma cells and non-parenchyma cells constituting liver.

More specifically, the present invention provides low density lipoprotein that secures blood stability by mimicking in vivo metabolism of endogenous low density lipoprotein through nanoparticles mimicking naturally existing low density lipoprotein and introducing lipo-PEG (polyethyleneglycol) complex, for example, DSPS-PEG (1,2-Distearoyl-phosphatidyl ethanolamine-methyl-polyethyleneglycol conjugate) in the layered structure of the nanoparticles, and has highly efficient liver cell (including parenchyma cells and non-parenchyma cells)—specific intracellular uptake property by electrostatic attraction between the cells and the nanoparticles by cationic lipids such as DC-Chol and destabilization of cell membrane mediated by phospholipids such as DOPE.

The nanoparticles of the present invention may highly efficiently deliver therapeutics in the liver cells through the cell specific uptake for the parenchyma cells and non-parenchyma cells of liver, and thus, the present invention also provides a method of delivering drugs for treatment of liver disease, nucleic acid gene for treatment and the like mediated by the highly efficient liver cell specific lipoprotein-like nanoparticles, and liver disease treatment technology using the same. Furthermore, the present invention provides non-invasive imaging diagnosis technology by adding the nanoparticles in the solid lipid core or as a lipid constituting ingredient of a composition for imaging diagnosis.

The treatment for which the nanoparticles of the present invention can be applied may include treatment of all kinds of liver disease, and more specifically, it may be 1) treatment of hepatotropic viral infection including 1) hepatitis B virus (HBV), hepatitis C virus (HCV), 2) treatment of acute and chronic liver fibrosis and liver cirrhosis occurred by hepatotropic viral infection, 3) treatment of various acute and chronic liver diseases including hepatic fibrosis, cirrhosis, acute liver failure due to drugs, alcohols and non-alcoholic steatohepatitits and the like. Wherein, substances delivered for treatment of liver disease by the nanoparticles may be liver specific high efficient drugs, nucleic acid genes, and the like.

In case the substance delivered by the nanoparticles is nucleic acid, viral replication and expression occurred in liver cells including parenchymal cells and non-parenchymal cells by hepatotropic viral infection including HBV, HCV may be inhibited to treat acute and chronic infection. And, for various acute and chronic liver diseases and hepatic fibrosis and cirrhosis induced thereby, the expression of various pro-fibrotic molecules and inflammatory cytokines including CTGF (connective tissue growth factor) expressed by effector cell population(s) (namely, hepatic stellate cell) may be controlled to treat liver diseases.

In the present invention, lipid-like nanoparticles reconstructed by mimicking the lipid construction of endogenous lipoprotein very similarly mimics metabolism of natural lipoprotein, which was confirmed to be through acquisition of apolipoprotein including apolipoprotein E that is involved in recognition and cellular uptake by lipid receptors such as low density lipoprotein receptor (LDLr) or remnant receptor of lipoprotein in the lipid metabolism process by liver cells including parenchyma cells and non-parenchyma cells.

Such acquisition of apolipoprotein by low density lipoprotein-like nanoparticles may be naturally induced from blood plasma and HDL (High-density lipoprotein) and the like after a complex of the nanoparticles and drugs or nucleic acid is injected in the blood, or it may be artificially induced through cultivation of the complex with apolipoprotein for a predetermined time. Wherein the apolipoprotein may be recombinant apolipoprotein E, detergent solubilised apoB-100 and the like, but is not limited thereto.

Hereinafter, the present invention will be explained in detail

One embodiment of the invention relates to a low density lipoprotein (LDL)-like nanoparticle with a core-shell structure comprising a core containing cholesteryl ester and triglyceride; and a cationic shell containing cholesterol, fusogenic lipids, cationic lipids, and a lipid-PEG (polyethyleneglycol) conjugate. The nanoparticle may be cationic solid nanoparticle (CSLN). The nanoparticle is reconstructed by mimicking the constitutional elements of natural low density lipoprotein, and it has excellent biocompatibility.

The shell is bonded to the top layer of the core by hydrophobic interaction, and includes exposed cationic lipids that can interact electrostatically with drugs, particularly anionic drugs and/or nucleic acid genes. The nanoparticles of the present invention may bind with drugs, particularly anionic drugs and/or nucleic acid genes by electrostatic interaction through the exposed cationic lipids of the shell, thus easily forming a complex, and it may be usefully used as a composition for intracellular delivery of drugs, particularly anionic drugs and/or nucleic acid genes.

The lipophilic core may encapsulate hydrophobic drugs, or hydrophobic or surface modified diagnosis composition in the core in a solid state at room temperature and body temperature through hydrophobic interaction in the preparation process of nanoparticles. If the encapsulated composition is hydrophobic drug, the particles may be released or controlled-released in the cells through diffusion and the like in the process of mimicking metabolism of low density lipoprotein, and if the encapsulated composition is quantum dot (Q.dot) or lipid-coated iron oxide particles, the particles may be usefully used for diagnosis through optical imaging, or MRI imaging.

And, by incorporating radioisotope labeled lipids in the shell of the particle, the particles may be usefully used for acquisition of diagnostic image such as PET or SPECT and the like.

Thus, another embodiment of the invention provides a composition for delivery of drugs and/or nucleic acid genes or a composition for treatment and imaging diagnosis, comprising the low density lipoprotein-like nanoparticles with a core-shell structure as an active ingredient.

Still another embodiment provides a complex of drugs or nucleic acid and low density lipoprotein-like nanoparticles, comprising the low density lipoprotein-like nanoparticles, and drugs or nucleic acid bonded to cationic lipid on the surface of the nanoparticles by electrostatic interaction.

The cholesteryl ester means cholesterol to which saturated or unsaturated fatty acid having a carbon number of 10 to 24 is ester-bonded. Preferably, it may be ester of unsaturated fatty acid having a carbon number of 16 to 18 such as oleic acid. The nanoparticles of the present invention may comprise various kinds of cholesteryl ester.

The triglyceride may be purified triglycerides having various fatty acid compositions, or vegetable oil containing triglyceride consisting of plural fatty acids as a main ingredient. Specifically, the triglyceride may be animal or vegetable oil, and it may be at least one selected from the group consisting of soybean oil, olive oil, cotton seed oil, sesame oil, liver oil and the like. The oil may be used in one kind or in the mixture of various kinds. According to specific embodiment, the triglyceride may be triolein.

The cholesteryl ester and triglyceride forms a core of a low density lipoprotein-like nanoparticle through hydrophobic bonding.

The fusogenic lipid may include all kinds of neutral, cationic or anionic lipids that can form the nanoparticle of the present invention, and it may be a single kind or a mixture of plural kinds of phospholipids. The fusogenic lipid may include all kinds of phospholipids that can induce fusion, and for example, it may be phosphatidylcholine (PC), phosphatidylethanolamine, phosphatidylserine, phosphatidylglycerol, or lyso forms thereof, or completely saturated or partially cured forms thereof having a C6-24 aliphatic chain. Specifically, the fusogenic lipid, although not specifically limited, may be at least one selected from the group consisting of dioleoylphosphatidylethanolamine (DOPE), palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), distearoylphosphatidylethanolamine (DSPE), phosphatidylethanolamine (PE), dipalmitoylphosphatidylethanolamine, 1,2-dioleyl-sn-glycero-3-phosphoethanolamine, 1-palmitoyl-2-oleyl-sn-glycero-3-phophoethanolamine (POPE), 1-palmitoyl-2-oleyl-sn-glycero-3-phosphocholine (POPC), 1,2-dioleyl-sn-glycero-3-[phospho-L-serine](DOPS), and 1,2-dioleyl-sn-glycero-3-[phospho-L-serine], and the like.

The fusogenic lipid and cholesterol constituting the shell improve transfection efficiency, and functions as helper lipid for decreasing cytotoxicity of combined cationic lipid. And, cholesterol affords firmness in terms of a shape, thus improving the stability of the nanoparticles of the present invention together with the activity of helper. And, the fusogenic lipid aids in passage through cell membrane and endosomal escape of the nanoparticles to facilitate intracellular delivery.

The cationic lipid includes cationic lipids entirely negatively charged at a specific pH such as physiological pH. Specifically, the cationic lipid may be at least one selected from the group consisting of 3beta-[N—(N′,N′,N′-trimethylaminoethane)carbomoyl]cholesterol (TC-cholesterol), 3beta[N—(N′,N′-dimethylaminoethane)carbamoyl]cholesterol (DC-cholesterol), 3beta[N—(N′-monomethylaminoethane)carbamoyl]cholesterol (MC-cholesterol), 3beta[N-(aminoethane)carbamoyl]cholesterol (AC-cholesterol), N—(N′-aminoethane)carbamoylpropanoic tocopherol (AC-tocopherol), N—(N′- [symbol] aminoethane)carbamoylpropanoic tocopherol (MC-tocopherol), N,N-dioleyl-N,N-dimethylammoniumchloride (DODAC), N,N-distearyl-N,N-dimethylammoniumbromide (DDAB), N-(1-(2,3-dioleoyloxy)propyl-N,N,N-trimethylammoniumchloride (DOTAP), N,N-dimethyl-(2,3-dioleoyloxy)propylamine (DODMA), N-(1-(2,3-dioleyl)propyl)-N,N,N-trimethylammoniumchloride (DOTMA), 1,2-dioleyl-3-dimethylammonium-propane (DODAP), 1,2-dioleylcarbamyl-3-dimethylammonium-propane (DOCDAP), 1,2-dilineoyl-3-dimethylammonium-propane (DLINDAP), dioleoyloxy-N-[2-sperminecarboxamido)ethyl}-N,N-dimethyl-1-propanaminium trifluoroacetate (DOSPA), dioctadecylamidoglycyl spermine (DOGS), 1,2-dimyristyloxypropyl-3-dimethyl-hydroxyethyl ammonium bromide (DMRIE), 3-dimethylamino-2-(cholest-5-en-3-beta-oxybutane-4-oxy)-1-(cis,cis-9,12-octadecadienoxy)propane (CLinDMA), 2-[5′-(cholest-5-en-3-beta-oxy)-3′-oxapentoxy]-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-diacyl-3-trimethylammonium-propane (TAP), 1,2-diacyl-3-dimethylammonium-propane (DAP), 1,2-di-O-octadecyl-3-trimethylammonium propane, and 1,2-dioleyl-3-trimethylammonium propane, Trasfectam®, 98N12-5(1), and the like.

Particularly, since DC-cholesterol has weaker toxicity than other cationic lipids, and DC-cholesterol based genetic carriers are accepted for use in clinical treatment of various diseases including melanoma, cystic fibrosis, uterine cervical cancer, breast cancer and ovarian cancer and the like, it may be preferable to use DC-cholesterol.

The lipid-PEG (polyethyleneglycol) conjugate means a conjugated form of lipid and PEG.

The lipid in the conjugate may be selected from the group consisting of the above explained all kinds of cholesterols, fusogenic lipids and cationic lipids, and for example, it may be lipid containing an amine group. According to specific embodiments, the lipid may be at least one selected from the group consisting of cholesterol, dioleoylphosphatidylethanolamine (DOPE), palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), distearoylphosphatidylethanolamine (DSPE), phosphatidylethanolamine (PE), dipalmitoylphosphatidylethanolamine (DPPE), 1,2-dioleoyl-sn-glycero-3-phosphoethanolamine, 1-palmitoyl-2-oleoyl-sn-glycero-3-phosphoethanolamine (POPE), 1-palmitoyl-2-oleoyl-sn-glycero-3-phosphocholine (POPC), 1,2-dioleoyl-sn-glycero-3-[phospho-L-serine] (DOPS), 1,2-dioleoyl-sn-glycero-3-[phospho-L-serine], 3beta-[N—(N′,N′,N′-trimethylaminoethane)carbomoyl]cholesterol (TC-cholesterol), 3beta[N—(N′,N′-dimethylaminoethane)carbamoyl]cholesterol (DC-cholesterol), 3beta[N—(N′-monomethylaminoethane)carbamoyl]cholesterol (MC-cholesterol), 3beta[N-(aminoethane)carbamoyl]cholesterol (AC-cholesterol), N—(N′-aminoethane)carbamoylpropanoic tocopherol (AC-tocopherol), N—(N′- [symbol] aminoethane)carbamoylpropanoic tocopherol (MC-tocopherol), N,N-dioleyl-N,N-dimethylammoniumchloride (DODAC), N,N-distearyl-N,N-dimethylammoniumbromide (DDAB), N-(1-(2,3-dioleoyloxy)propyl-N,N,N-trimethylammoniumchloride (DOTAP), N,N-dimethyl-(2,3-dioleoyloxy)propylamine (DODMA), N-(1-(2,3-dioleyl)propyl)-N,N,N-trimethylammoniumchloride (DOTMA), 1,2-dioleyl-3-dimethylammonium-propane (DODAP), 1,2-dioleylcarbamyl-3-dimethylammonium-propane (DOCDAP), 1,2-dilineoyl-3-dimethylammonium-propane (DLINDAP), dioleoyloxy-N-[2-sperminecarboxamido)ethyl}-N,N-dimethyl-1-propanaminium trifluoroacetate (DOSPA), dioctadecylamidoglycyl spermine (DOGS), 1,2-dimyristyloxypropyl-3-dimethyl-hydroxyethyl ammonium bromide (DMRIE), 3-dimethylamino-2-(cholest-5-en-3-beta-oxybutane-4-oxy)-1-(cis,cis-9,12-octadecadienoxy)propane (CLinDMA), 2-[5′-(cholest-5-en-3-beta-oxy)-3′-oxapentoxy]-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-diacyl-3-trimethylammonium-propane (TAP), 1,2-diacyl-3-dimethylammonium-propane (DAP), 1,2-di-O-octadecyl -3-trimethylammonium propane, 1,2-dioleyl-3-trimethylammonium propane, Trasfectam®, 98N12-5(1), and the like. Specifically, the lipid may be distearoylphosphatidylethanolamine (DSPE), dipalmitoylphosphatidylethanolamine (DPPE) and the like, but is not limited thereto.

The PEG, although not specifically limited, may have a weight average molecular weight of 100 to 100,000 Daltons, specifically 500 to 70,000 Daltons, more specifically 1,000 to 50,000 Daltons. According to one specific embodiment, PEG may be functionalized PEG, wherein the functional groups that can be used may be at least one selected from the group consisting of a succinyl group, carboxylic acid, maleimide, an amine group, biotin, cyanur, folate and the like.

The mole ratio of the lipid and the PEG in the lipid-PEG conjugate may be about 1:0.5 to 3 (lipid moles:PEG moles).

According to specific embodiments, the lipid-PEG conjugate may be distearoylphosphatidylethanolamine (DSPE)-PEG or dipalmitoylphosphatidylethanolamine (DPPE)-PEG, and the like. The lipid-PEG conjugate contributes to serum stability of the nanoparticles, and functions for protecting nucleic acid from degradation enzyme during in vivo delivery of nucleic acid, thus increasing in vivo stability of nucleic acid.

According to specific embodiments, the nanoparticle may be low density lipoprotein-like nanoparticle comprising a core containing cholesteryl oleate and triolein; and a shell containing cholesterol, dioleylphosphatidylethanolamine (DOPE), 3β[N—(N′,N′-dimethylaminoethane)carbamoyl]-cholesterol (DC-cholesterol), and DSPE-PEG.

The cationic lipid located in the shell of the nanoparticle may bind with drugs, particularly anionic drugs, and/or nucleic acid through electrostatic bonding to form a complex.

The nanoparticle of the present invention is low density lipoprotein-like (LDL-like) cationic nanoparticle. Naturally existing LDL consists of two lipid phase, i.e., a polar constituting part (phospholipid and apolipoprotein) and non-polar neutral lipid previously consisting of cholesteryl ester and triglyceride, and has the composition and physicochemical properties as shown in the following Table 1. The phospholipid and apolipoprotein emulsify the non-polar lipids to afford surface stability, thus forming stabilized biological microemulsion.

TABLE 1

| | Natural LDL | |
|---|---|---|
| | Constitutional | Contents |
| | elements | (w/w) |
| Core | Cholesteryl ester | 45% |
| | Triglyceride | 3% |
| Surface | Cholesterol | 10% |
| | Phospholipid | 22% |
| | Apolipoprotein B-100 | 20% |
| Size (nm) | 18-25 | |
| Zeta potential (mV) | −11.4 ± 1.9 [25] | |

According to specific embodiments, the LDL-like nanoparticle comprises 30 to 60 wt % of cholesteryl ester, 0.1 to 10 wt % of triclyceride, 5 to 20 wt % of cholesterol, 5 to 30 wt % of fusogenic lipid, 10 to 50 wt % of cationic lipid, and 0.01 to 1 wt % of lipid-PEG conjugate, based on the total weight of nanoparticle. Preferably, it comprises 40 to 50 wt % of cholesteryl ester, 1 to 5 wt % of triclyceride, 8 to 12 wt % of cholesterol, 12 to 16 wt % of fusogenic lipid, 25 to 30 wt % of cationic lipid, and 0.05 to 5 wt % of lipid-PEG conjugate.

If the content of the lipid-PEG conjugate in the nanoparticle is greater than the above range, it may be difficult to form a complex with nucleic acid, and if it is less than the above range, it is unfavorable in terms of serum stability of the particles and in vivo stability of nucleic acid, and thus, considering easiness of formation of a complex of nanoparticles and nucleic acid, in vivo stability of nanoparticles, in vivo stability of nucleic acid and the like, it is preferable that the content of the lipid-PEG conjugate may be within the above range.

And, the content ratio of the core and the shell in the nanoparticle of the present invention may be 30:70 to 70:30, specifically 40:60 to 60:40, more specifically 45:55 to 55:45 by weight.

According to one embodiment of the invention, the mole ratio of the fusogenic lipid:cholesterol:cationic lipid in the shell constituting components may be 9.4:13:26, and the mole ratio of cationic lipid/helper lipid (fusogenic lipid and cholesterol) may be 1.16, which provides equimolar ratio.

The LDL-like nanoparticle of the present invention may have an average particle diameter of 70 nm to 110 nm so as to be easily introduced in liver cells.

According to specific embodiment, the LDL-like nanoparticle may further comprise at least one selected from the group consisting of hydrophobically surface modified particles, for example, hydrophobically surface modified quantum dot, carbone dot, gold nanoparticles, iron oxide nanoparticles and the like. As such, by further including hydrophobically surface modified particles, the DLD-like nanoparticles may be easily imaged and thus more favorably applied for diagnosis field. Wherein, the hydrophobically surface modified particles that are further included may have an average diameter of 2 nm to 50 nm, for example, 2 nm to 20 nm, but is not limited thereto. The content of the hydrophobically surface modified particles may be 1% (w/w) to 20% (w/w), based on the total weight of nanoparticles (the weight of nanoparticles including hydrophobically surface modified particles).

According to another embodiment, the shell of the LDL-like nanoparticle may further comprise radioisotope labeled lipids obtained by radioisotope labeling of the above explained shell constituting elements, namely, at least one selected from the group consisting of cholesterol, fusogenic lipid, cationic lipid and lipid-PEG conjugate. By including radioisotope labeled lipids in the shell of the nanoparticle, the nanoparticle may be easily imaged and thus more favorably applied for diagnostic field. Wherein, the radioisotope that can be used may be selected from the group consisting of P32, C11, H3, O15, N13 and the like, and the amount of further included radioisotope labeled lipids may be 0.001 to 5% (w/w) of the total weight of the LDL-like nanoparticles (the weight of the nanoparticles including radioisotope labeled lipids). In case the radioisotope is labeled on lipid-PEG conjugated, it may be labeled on a functional group of lipid capable of binding or a functional group of PEG Wherein, even after adding the radioisotope labeled lipids, the amount of added radioisotope labeled lipids may be controlled within the above range, so that the ratio of the shell and the core may be within the above described range.

The LDL-like nanoparticle of the present invention may be used for transportation of physiological active substance such as anionic or hydrophobic drugs and/or nucleic acids by electrostatic interaction with exposed cationic lipid of the shell.

The nucleic acid may be at least one selected from the group consisting of small interfering RNA (siRNA), ribosomal ribonucleic acid (rRNA), ribonucleic acid (RNA), deoxyribonucleic acid (DNA), complementary DNA (cDNA), aptamer, messenger ribonucleic acid (mRNA), transfer ribonucleic acid (tRNA), and antisense oligodeoxynucleotide (AS-ODN), and the like, but is not limited thereto. Nucleic acid may be bonded with the cationic lipid of the nanoparticle by electrostatic interaction and included in a complex.

Particularly, siRNA refers to duplex RNA, or single stranded RNA that takes the form of double strand in single stranded RNA. Bonding between double strands is achieved through hydrogen bond between nucleotides, and all the nucleotides in double strands should not complementarily bind. The length of siRNA may be about 15 to 60, specifically about 15 to 50, about 15 to 40, about 15 to 30, 15 to 25, about 16 to 25, about 19 to 25, about 20 to 25, or about 20 to 23 nucleotides. The length of siRNA means the number of nucleotides of one of double strand RNA, i.e., the number of base pairs, and for single strand RNA, it means the length of double strand in single stranded RNA. And, siRNA may consist of various functional groups-introduced nucleotides so as to increase blood stability or weaken immune reactions.

Thus, the siRNA of the present invention may be non-modified or modified form of typical siRNA. For example, one end of siRNA may be modified with polyethyleneglycol. Since polyethyleneglycol (PEG) is hydrophilic, flexible and non-ionic polymer, it is one of common materials for modifying particle system so as not to be recognized by macrophage of unimolecular macrophage system (MPS) and affording long-term circulation to a carrier. According to one embodiment of the invention, if the molecular weight of PEG is 3000 to 7000 Daltons, for example 5000 Daltons, it may sufficiently protect siRNA to RNase digestion while maintaining effective transfection performance of siRNA.

The anionic drugs may be anionic biopolymer-drug conjugate such as negatively charged peptides, protein drugs or hyaluronic acid-peptide conjugate, hyaluronic acid-protein conjugate and the like. The hydrophobic drug may be taxol, doxorubicin, epirubicin and the like. The drug may be encapsulated in the core of the LDL-like nanoparticle.

The drug-LDL-like nanoparticle complex or nucleic acid-LDL-like nanoparticle complex may further comprise at least one selected from the group consisting of hydrophobically surface modified detection particles, for example, hydrophobically surface modified quantum dot, carbone dot, gold nanoparticles, iron oxide nanoparticles and the like or it may further comprise radioisotope labeled lipids in the shell. In this case, it is advantageous in that delivery of hydrophobic or anionic drugs or nucleic acids and imaging by hydrophobically surface modified detection particles may be simultaneously progressed.

The weight ratio of the LDL-like nanoparticles to the weight of nucleic acids (based on the weight of 21 mer siRNA) (LDL-like nanoparticle weight/nucleic acid weight) in the nucleic acid-LDL-like nanoparticle complex of the present invention may be 10 to 50, specifically 20 to 40, or 25 to 35, for example, about 30.

The LDL-like nanoparticle of the present invention may comprise one kind or plural kinds of apoprotein. The apoprotein may be extracted from natural lipoprotein or producted by a method for recombinant protein, and preferably, it may be B-100, apo E and the like. The apoprotein may enable the nanoparticles of the present invention to be effectively and specifically introduced in the cells through a receptor.

The target cells to which drugs and/or nucleic acids are delivered by the LDL-like nanoparticles of the present invention may be liver cells including parenchyma cells and non-parenchyma cells in the body or separated from the body. Thus, a composition for delivery of drugs and/or nucleic acids comprising the LDL-like nanoparticles of the present invention, and a complex of drugs and/or nucleic acids and LDL-like nanoparticles may specifically target liver cells (parenchyma cells and non-parenchyma cells). Thus, the nanoparticles according to the present invention or a composition for delivery of drugs and/or nucleic acids comprising the nanoparticles may be used for treatment of acute or chronic liver diseases such as hepatic fibrosis, cirrhosis, hepatitis (for example, hepatitis A, hepatitis B, hepatitis C and the like), and the like.

According to yet another aspect of the invention, provided is a method for delivery of drugs and/or nucleic acids to target cells using the LDL-like nanoparticles, and one embodiment provides a method for liver specific delivery of anionic or hydrophobic drugs or nucleic acids, comprising the step of administering the composition for delivery to a subject.

The subject may be mammals, for example, human being. The route of administration may be intravascular, intramuscular, subcutaneous, oral, bone, transdermal, topical tissue, and the like, but is not limited thereto.

According to specific embodiment, the method may comprise the steps of (1) forming a complex of drugs and/or nucleic acids and the LDL-like nanoparticles; and (2) transfecting target cells with the complex salt.

The complex of the step (a) may be formed, for example, by mixing the LDL-like nanoparticles with drugs and/or nucleic acids in phosphate buffered saline (PBS) or deionized water, wherein the PBS preferably has pH of 7.0˜8.0 and contains 0.8% (w/v) of NaCl, but not limited thereto.

In case nucleic acid is used, it may be in the form of PEG modified nucleic acid-PEG conjugate. In this case, after the step (a), a step of gel delay treating the nucleic acid-PEG conjugate and the LDL-like nanoparticle complex may be further included. The gel delay treatment is conducted to confirm whether negatively charged nucleic acid (for example, siRNA) and positively charged nanoparticles (cationic lipid of shell) form a stabilized complex by electrostatic interaction therebetween.

In case PEG-modified nucleic acid is used, before the step (1), a step of (1′) forming a nucleic acid-PEG conjugate may be further included.

Specifically, the conjugate of the step (1′) may use, for example, a disulfide bond between nucleic acid and PEG, and more specifically, the nucleic acid preferably has a 3′-hexylamine functional group. On PBS (Ph 7.5), excessive SPDP (N-succinimidyl-3-(2-pyridylodithio)propionate) reacts with 3′-hexylamine siRNA to activate nucleic acid. Non-reacted SPDP is removed using a desalting column The siRNA activated by SPDP reacts excessively with polyethyleneglycol (PEG, molecular weight:5000)-SH to form a disulfide bond and is conjugated with PEG. Non-reacted PEG-SH is removed by dialysis (MWCO=10000) to purify siRNA conjugated with PEG by disulfide bonds.

For example, although siRNA is powerful means for use in genetic treatment due to preferable inhibition of gene expression, it has limitations in practical application due to the problems relating to stability and transfection effect.

Since the cationic lipid nanoparticles (CLM) of the present invention forms a stable complex with nucleic acid through electrostatic interaction in serum-containing medium, and the CLM exhibits extremely low cytotoxicity and effective cellular uptake together with nucleic acid, it is effective for delivering nucleic acid. And, serum apolipoprotein may be adsorbed on the surface of CLM due to high blood affinity and mimic natural apolipoprotein-containing lipoprotein. Thus, body CLM is assumed to be produced by the action of natural lipoprotein.

According to specific embodiment, provided is a composition for imaging comprising a core containing cholesteryl ester and triglyceride; a shell containing cholesterol, fusogenic lipid, cationic lipid, and a lipid-PEG (polyethylneglycol) conjugate; and nanoparticles including hydrophobically surface modified particles (for example, at least one selected from the group consisting of hydrophobically surface modified quantum dot, carbone dot, gold nanoparticles, iron oxide nanoparticles and the like). According to another embodiment, provided is a composition for imaging, comprising a core containing cholesteryl ester and triglyceride; and a shell containing cholesterol, fusogenic lipid, cationic lipid, lipid-PEG (polyethyleneglycol) conjugate, and radioisotope labeled lipids obtained by radioisotope labeling of at least one selected from the group consisting of cholesterol, fusogenic lipid, cationic lipid and lipid-PEG conjugate. The imaging composition may be liver specific. The size and content of the hydrophobically surface modified particles, the content of the radioisotope labeled cationic lipids replacing the cationic lipids, and the kind of the radioisotope are as explained above.

According to another aspect of the invention, provided is a method for detecting an imaging composition, and one embodiment comprises the steps of administering the imaging composition to a subject; and detecting the imaging composition.

According to specific embodiment, the method may comprise administering to a subject a liver specific imaging composition comprising a core containing cholesteryl ester and triglyceride; a shell containing cholesterol, fusogenic lipid, cationic lipid, and a lipid-PEG (polyethylneglycol) conjugate; and nanoparticles including at least one kind of hydrophobically surface modified particles selected from the group consisting of hydrophobically surface modified quantum dot, carbone dot, gold nanoparticles, iron oxide nanoparticles and the like; and detecting the imaging composition, and according to another embodiment, the method may comprise administering to a subject a liver specific imaging composition comprising a core containing cholesteryl ester and triglyceride; and a shell containing cholesterol, fusogenic lipid, cationic lipid, lipid-PEG (polyethyleneglycol) conjugate, and radioisotope labeled lipid obtained by radioisotope labeling of at least one selected from the group consisting of cholesterol, fusogenic lipid, cationic lipid and lipid-PEG conjugate; and detecting the imaging composition.

The subject may be mammals, for example, human being. The route of administration may be intravascular, intramuscular, subcutaneous, oral, bone, transdermal, topical tissue and the like but is not limited thereto.

The detection step may be conducted by known detection methods. For example, in case the imaging composition comprises quantum dot or iron oxide, it may be detected by optical imaging or MRI imaging, and thus, it may be usefully used for diagnosis. In case the imaging composition comprise radioisotope, it may be usefully used for acquisition of diagnostic image using PET or SPECT and the like.

### Advantageous Effects

The LDL-like nanoparticles of the present invention mimics the constituents of naturally existing low density lipoprotein, and thus, it is biodegradable, does not induce an immune reaction, and effectively blocks non-specific elimination by reticuloendothelial system, thus providing very stable nanoparticle-based treatment technology.

And, by mimicking the metabolism of natural lipoprotein, it may deliver useful bioactive materials such as cell specific drugs, nucleic acids and the like to liver cells including parenchyma cells and non-parenchyma cells.

And, due to the liver cell-specific targeting of the LDL-like nanoparticles, it may be useful for treatment of acute or chronic intractable liver diseases.

### BRIEF DESCRIPTION OF THE DRAWING

FIG. 1 schematically shows a low density lipoprotein-like nanoparticle according to one embodiment (hereinafter ‘CSLN’) and shows the comparison with the composition of natural low density lipoprotein-like nanoparticle.

FIG. 2 is a height AFM image of CSLN according to one embodiment.

FIG. 3 shows the result of comparing the cytotoxicity of CSLN according to one embodiment with that of PEI (control).

FIG. 4 is an image analyzed by electrophoresis of a complex of CSLN according to one embodiment and siRNA (CSLN/siRNA complex).

FIG. 5 shows the result of siRNA protection effects of naked siRNA and CSLN/siRNA complex according to one embodiment

FIG. 6 shows the result of siRNA protection effect of DSPE-PEG in the constituents of CSLN according to one embodiment, through comparison with CSLN without DSPE-PEG.

FIG. 7 shows the observation results after counter staining HepG2.2.15 cells that have been treated with CSLN/FAM-siRNA for 2 hours.

FIG. 8 shows four siRNAs (siHBV; si257, si1658, si1826 and si2422) targeting different 4 overlapping points of open reading frame of HBV virus genome used in Example 6.

FIG. 9 shows the results of HBsAg gene silencing obtained by treating Hepg2.2.15 cells with CSLN/siRNA complexes comprising different four siHBVs (si257, si1658, si1826 and si2422) and target regions on HBV genome.

FIG. 10 shows blood ALT, AST, TBIL, ALB levels obtained by analyzing the blood sample obtained in Example 8.

FIGS. 11a to 1ld show blood TNF-α, TGF-β, and IL-6 levels obtained by analyzing the blood sample obtained in Example 8, and the expression degrees of CTGF and tubulin in tissue analyzed using liver tissue (right bottom).

FIG. 12 shows the results of histologic and immunologic analysis of fibrotic liver tissue extracted from animals treated with the control described in Example 8, NDMA+CSLN/siCTGF, NDMA+CSLN/scCTGF, and NDMA+CSLN, wherein (a) shows the results of H&E staining of the samples obtained from each group, (b) shows the results of Masson's trichrome staining of the samples obtained from each group, and (c) shows the results of α-SMA (green), CTGF (red), and nucleus (blue) staining of the samples obtained from each group through immunofluorescent staining.

FIG. 13 shows the results of observation after intravascular administration of CSLN/FAM-siRNA complex according to one embodiment, followed by cryosection of left lever (left) and right lateral liver robe (right).

FIG. 14 shows the results of confirming biodistribution of CSNL-Q.Dot/siRNA complex over time through optical imaging after intravascular administration of CSNL-Q.Dot obtained in Example 11 in animal body.

FIG. 15 shows the results of confirming distribution of CSLN/siRNA over time according to organs, from the results of FIG. 14.

FIG. 16 shows the results of conducting X-ray liver imaging of in vivo behavior of SLN/siRNA comprising radioisotope labeled lipids and liver specific delivery and accumulation of CSNL.

### DETAILED DESCRIPTION OF THE EMBODIMENTS

The present invention will be explained in detail with reference to the following examples, but the scope of the invention is not limited thereto.

### EXAMPLE 1

### Preparation of Low Density Lipoprotein-Like Nanoparticle

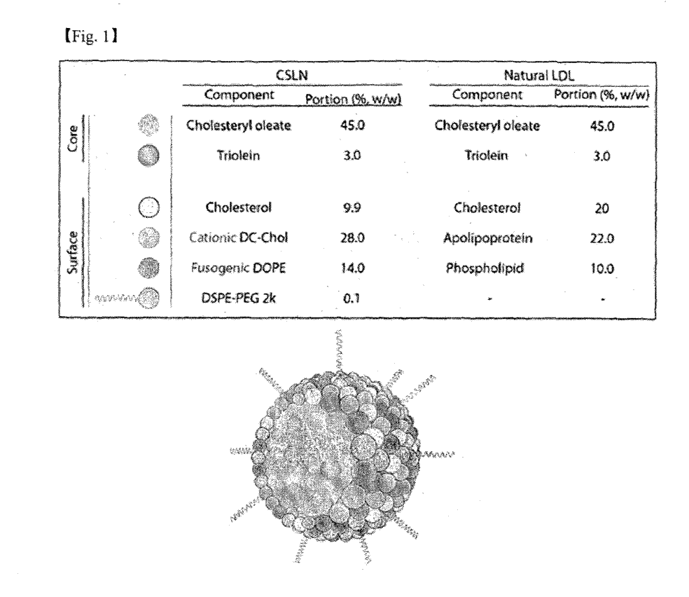

Cationic low density lipoprotein-like nanoparticle (hereinafter CSLN: cationic solid lipid nanoparticle) was prepared using the composition described in the Table below, and the schematic diagram of the nanoparticle comprising the composition and the comparison with the composition of natural low density lipoprotein-like nanoparticle are shown in FIG. 1.

TABLE 2
CSLN

| | Portion |
|---|---|
| Component | (%, w/w) |
| Cholesteryl oleate | 45.0 |
| Triolein | 3.0 |
| Cholesterol | 9.9 |
| Cationic DC-Chol | 28.0 |
| Fusogenic DOPE | 14.0 |
| DSPE-PEG 2 k | 0.1 |

The weight ratio of each lipid in the total weight of 50 mg of lipid constituents was 45.0% of cholesteryl oleate, 3.0% of triolein, 9.9% of cholesterol, 28.0% of cationic DC-cholesterol, 14.0% of fusogenic DOPE and 0.1% of DSPE-PEG 2K (1,2-distearoyl-sn-glycero-3-phosphoethanolamine-N-(polyethylene glycol)-2000), and the lipid constituents (50 mg) were dissolved in 2 ml of mixed solvents of chloroform and methanol at 2:1(chloroform:methanol (v/v)) put in a glass bottle. To the bottle, 10 mL of distilled water (DDDW) was added and the mixture was vortexed for 1 minute. The obtained suspension was ultrasonicated for 5 minutes (amplitude=35%, pulse on=5.0 sec, and pulse off=3.0 sec). The obtained suspension was transferred to a 100 ml round-bottom flask, followed by rotary evaporation at 60° C. to remove the solvents, and dialyzed with distilled water for use in the following experiments.

### EXAMPLE 2

### Dynamic Light Scattering

The CSLN suspension obtained in Example 1 was diluted to the concentration of 1 mg/ml using 0.1 M PBS (pH 7.4). For the obtained diluted suspension, dynamic light scattering was conducted using Malvern Zetasizer Nano ZS apparatus (Malvern, UK), thereby confirming the hydrodynamic size and the zeta potential of CSLN.

Thereafter, the suspension was mixed with siRNA (Connective tissue growth factor (CTGF) siRNA (siCTGF)) of the weight corresponding to 1/30 of the total weight of CSLN included in the diluted suspension, and the mixture was incubated at room temperature for 10 minutes.

siCTGF sense strand
(SEQ ID NO: 1)
5′-CAAUACCUUCUGCAGGCUGGAdTdT-3′
siCTGF antisense strand
(SEQ ID NO: 2)
5′-UCCAGCCUGCAGAAGGUAUUGdTdT-3′

For the obtained CSLN/siRNA mixed suspension, dynamic light scattering was conducted by the same method as above to confirm the hydrodynamic size and the zetapotential of CSLN/siRNA complex.

The obtained results are shown in the following Table 3.

TABLE 3

| | Hydrodynamic | |
|---|---|---|
| | diameter | Zeta potential |
| CSLN alone | 106.2 ± 5.4 nm | 64.3 ± 2.5 Mv |
| CSLN/siRNA complex | 113 ± 3.2 nm | 32.1 ± 3.3 mV |

The Table 3 shows the results of dynamic light scattering analysis of the CSLN/siRNA polyelectrolyte complex obtained by mixing the CSLN suspension prepared in Example 1 and siRNA, and it shows that the CSLN has a narrow size distribution and net positively charged by the cationic DC-Chol included in the constituents. And, through the results that the hydrodynamic diameter of the CSLN/siRNA complex obtained by mixing cationic CSLN and anionic siRNA increase, while the zeta potential value decreases, it is confirmed that a polyelectrolyte complex is formed through electrostatic interaction between cationic CSLN and anionic siRNA.

### EXAMPLE 3

### Analysis of Characteristics of CSLN

3.1. AFM Analysis

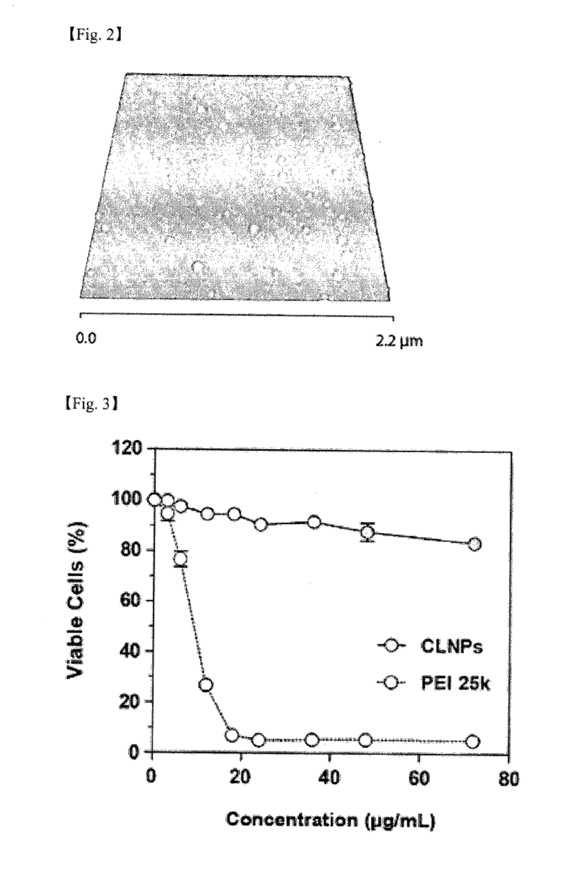

The CSLN suspension prepared in Example 1 was diluted to the concentration of 0.1 mg/ml using distilled water. 700 μl of the diluted suspension was dropped onto a mica plate of 1 cm×1 cm size. The mica plate onto which the diluted CSLN was dropped was dried in a dessicator for 12 hours. For the dried mica plate, tapping mode AFM analysis was conducted using Multimode SPM apparatus of Veeco Company, to confirm the topology of CSLN. The obtained results are shown in FIG. 2. The FIG. 2 is a height AFM image of the CSLN, and it shows that CSLN forms stable spherical assemblies properly dispersed within a diameter range of 102±2.4 nm on the average.

3.2. Evaluation of Cytotoxicity of CSLN

After seeding HepG2 cell line (Korean Cell Line Bank, KCLB No 88065) on a 48-well plate at 2×10⁴ cells/well, it was maintained in MEM culture medium (GIBCO Invitrogen, Carlsbad, Calif.) containing 1% (w/v) antibiotics and 10% (v/v) FBS for 24 hours (37° C., 5% CO₂). Thereafter, the cells were exposed to CSLN suspension prepared at the concentration of 0-72 μg/ml or PEI 25k (control) and incubated at 37° C. for 10 hours. Thereafter, CSLN-or PEI-containing culture medium was removed, and the cells were washed with PBS (pH 7.4) three times. Thereafter, the cells were incubated with 10 ul of Ez-cytotox reagent (Daeillab Service Co. Ltd., Korea) at 37° C. for 1 hour. And then, absorbance was measured at a wavelength of 450 nm using a micro-plate reader, and cytotoxictities of CSLN and PEI 25 k were respectively evaluated by comparing the absorbance.

The obtained results are shown in FIG. 3. The FIG. 3 shows the results of comparing cytotoxicity of CSLN with PEI (control) that is mostly used as a cationic polymer nucleic acid gene carrier, wherein CSLN exhibits extremely low cytotoxicity in the whole test concentration range, while PEI rapidly exhibits cytotoxicity from the low concentration, just 30% or less of cells can survive at about 10 μg/ml, and just about 5.1% of cells can survive at the concentration of 72 μg/ml, thus exhibiting high cytotoxicity. The IC50 values of CSLN and PEI 25k are respectively 333.5 μg/mL and 6.5 μg/ml, which show excellent biocompatibility and remarkably low cytotoxicity of CSLN.

3.3. Formation of CSLN/siRNA Complex

The CSLN suspension prepared in Example 1 was diluted with 0.1 M PBS to prepare diluted suspension samples containing 10-35 ug of lipids, which were respectively mixed with 1 ug of siRNA on 0.1 M PBS (Ph 7.4). The mixed suspension was incubated at room temperature for 10 minutes. The obtained mixed suspension was loaded on 2% agarose gels containing EtBr, and then, electrophoresed at 100 v for about 5 minutes. The obtained agarose gel was imaged under UV illuminator, and then, the migration degree of siRNA was analyzed to confirm the formation of a complex between siRNA and SCLN.

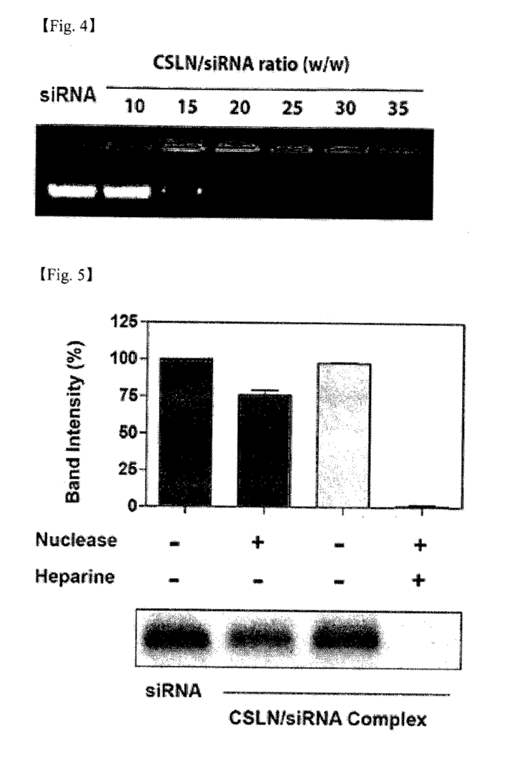

The obtained electrophoresis result is show in FIG. 4. The FIG. 4 is an electrophoresis image of CSLN/siRNA suspensions mixed at various weight ratios, and through the analysis of siRNA migration pattern shown in the image, it is confirmed that at the weight ratio (w/w) of CSLN/siRNA of about 30 or more, CSLN completely retards the migration of siRNA. This result shows that at the weight ratio of 30 or more, a polyelectrolyte complex is effectively formed by the electrostatic interaction between positively charged CSLN and negatively charged siRNA. And, from the result, it is confirmed that the PEG chain introduced on the surface of CSLN does not significantly inhibit electrostatic interaction between CSLN and siRNA or interfere formation of a complex.

### EXAMPLE 4

### siRNA Protection Effect

After preparing naked siRNA (5 μg) and a siRNA/CSLN complex comprising the same amount of siRNA at the weight ratio of CSLN/siRNA of 30 (prepared in Example 2) on 0.1 M PBS, they were mixed with 50 ul of reaction buffer (Promega, Madison, Wis.) containing 10 units of RNase ONE ribonuclease and incubated at room temperature for 1 hour.

After blocking RNA activity, it was incubated with 10 IU of heparin, and siRNA disintegrated from the complex was developed on 2% agarose gels containing EtBr. Thereafter, the agarose gel was observed on UV illuminator, and the result was demsitometrically analyzed to quantitatively analyze.

The obtained result is shown in FIG. 5. The FIG. 5 shows the siRNA protection effect of CSLN by directly treating naked siRNA or CSLN/siRNA complex with 10 units of nuclease, and shows the influence of nuclease on siRNA when the complex is treated simultaneously with nuclease and heparin and siRNA is separated from CSLN. The results show that siRNA dissociated from the complex by heparin treatment is completely decomposed by nuclease, while in case a complex is formed, about 75.16±3.3% of siRNA is protected intact.

From these results, it can be seen that the formation of a complex between CSLN and siRNA has the effect of protecting therapeutic nucleic acid genes (siRNA) intact from the attack of numerous nucleases existing in blood when injected in blood. And, by confirming the complete decomposition of siRNA in the group treated with heparin together with nuclease, it was confirmed that the bond between CSLN and siRNA is formed by electrostatic attraction between cationic CSLN and anionic nucleic acid genes.

### EXAMPLE 5

### Evaluation of PEG Effect

CSLN containing DSPE-PEG prepared in Example 1, and CSLN without DSPE-PEG prepared from the composition of Table 2 except DSPE-PEG were respectively prepared.

The prepared CSLN (w/PEG) and CSLN (w/o PEG) were respectively treated for 30 minutes under the same conditions as Example 4. Each obtained sample was electrophoresed by the same method as Example 4.

The results are shown in FIG. 6. The FIG. 6 shows the siRNA protection effect of DSPE-PEG in the constituents of CSLN through comparison with CSLN without DSPE-PEG, and it shows that the nucleic acid protection effect of CSLN as confirmed in Example 4 results from steric protection by hydrophilic PEG chain introduced on the surface of CSLN together with condensation of nucleic acid genes by cationic charge of CSLN.

### EXAMPLE 6

### Confocal Microscope Imaging

HepG2.2.15 cell line was seeded on a 24-well plate at 2×10⁴ cell/well, and maintained on MEM culture medium containing 10% (v/v) FBS and 200 μg/ml of a G418 antibiotic solution (Invitrogen, Carlsbad, Calif.) for 24 hours (37° C., 5% CO2). Thereafter, the cells were washed with PBS (pH 7.4) three times, and fresh MEM culture medium containing 10% FBS and 200 μg/ml of G148 antibiotics was supplied. The cultured cells were maintained in MEM culture medium containing 10% FBS, 2 μg/ml of G418 and a CSLN/FMA-siRNA complex (prepared by the method of Example 2) containing siRNA labeled with 25 nM of FAM (FMA-siRNA; siRNA with 21 mer TT overhang, Invitrogen, Carlsbad, Calif.) for 2 hours (37° C., 5% CO₂).

After incubation for 2 hours, the cells were washed with PBS three times, fixed with a 4% formaldehyde solution, and then, treated with a DAPI solution diluted to 1.5 ug/ml in PBS to counterstain nucleus, and imaged with confocal microscope.

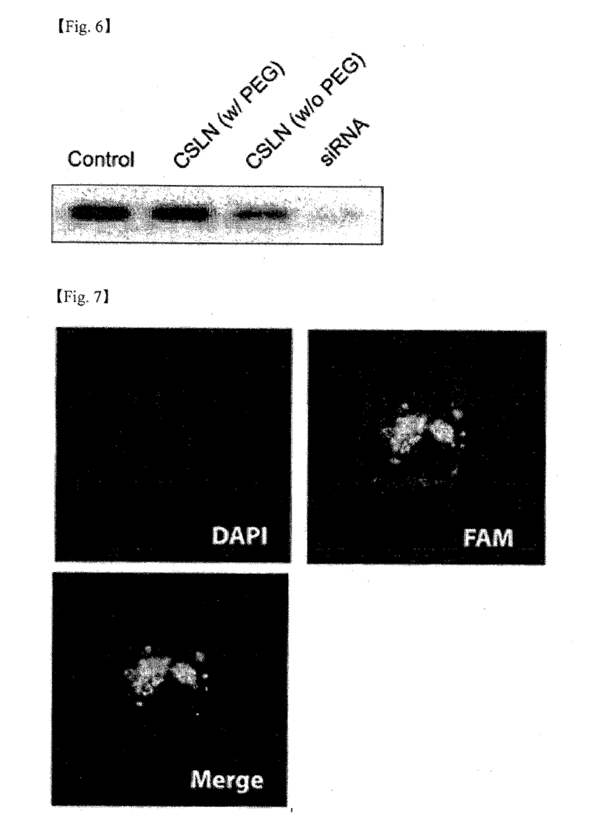

The results are shown in FIG. 7. FIG. 7 shows the result of observation after counter staining HepG2.2.15 cells treated with CSLN/FAM-siRNA for 2 hours with DAPI, and it shows that CSLN can internalize FAM-siRNA in the cytosol of the cells rapidly and effectively.

### EXAMPLE 7

### In Vitro Gene Silencing Effect using CSLN

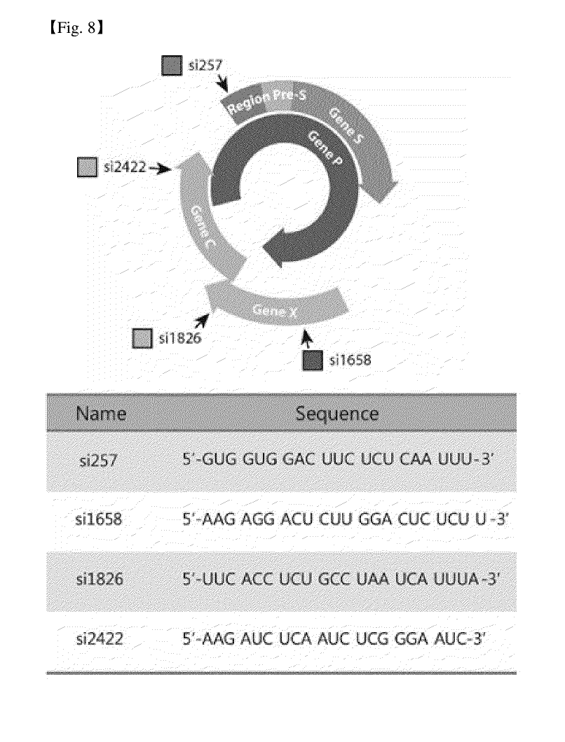

HepG2.2.15 cell line producing HBV RNA and virus particles was seeded on a 48-well plate at 4×10⁴ cell/well, and then, maintained on MEM culture medium containing 10% FBS and 200 μg/ml of a G418 antibiotic solution for 24 hours. After 24 hours, the cells were washed with PBS three times, and then, the culture medium was replaced with fresh MEM culture medium containing 10% FBS and 200 μg/ml of a G418 antibiotic solution. Thereafter, 4 kinds of siRNAs (si257, si658, si1826, si2422; hereinafter referred to as siHBV; see FIG. 8) respectively targeting different overlapping regions of HBV gene were mixed with CSLN to prepare CSNL/siHBV complexes by the method of Example 2.

si257 siRNA sense strand
(SEQ ID NO: 3)
5′-GUGGUGGACUUCUCUCAAUUU-3′
si658 siRNA sense strand
(SEQ ID NO: 4)
5′-AAGAGGACUCUUGGACUCUCUU-3′
si1826 siRNA sense strand
(SEQ ID NO: 5)
5′-UUCACCUCUGCCUAAUCAUUUA-3′
si2422 siRNA sense strand
(SEQ ID NO: 6)
5′-AAGAUCUCAAUCUCGGGAAUC-3′

The cultured cells were treated with the complex at the concentration of 0-200 Nm. After incubation for 5 hours (37° C., 5% CO₂), the culture medium was replaced with fresh MEM culture medium containing 10% FBS and 200μg/ml of a G418 antibiotic solution, and the cells were additionally incubated for 6 hours (37° C., 5% CO₂). After the incubation, the culture medium was replaced again, and the cells were additionally incubated for 48 hours (37° C., 5% CO₂), and then, HBsAg discharged from the cells was measured with ELISA kit (Green Cross Corp.). Relative HBsAg level was confirmed, based on the level of HBsAg discharged from non-siRNA-treated control cells as 100%.

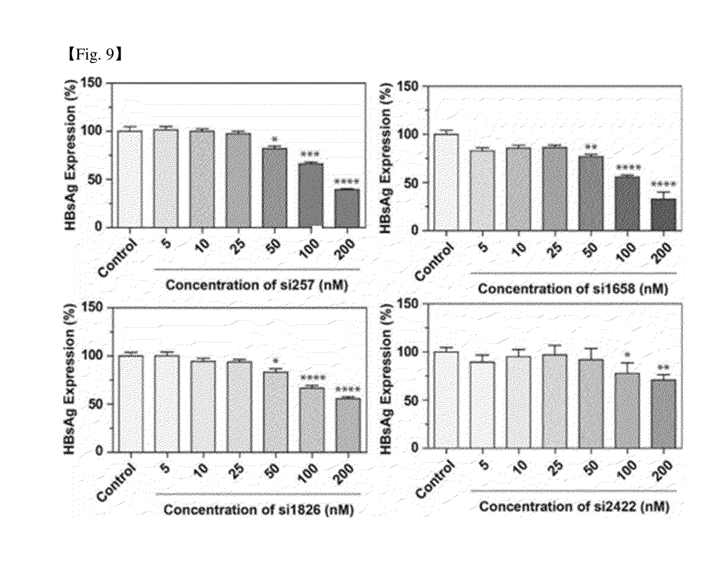

The results are shown in FIG. 9. FIG. 9 shows the results of HBsAg gene silencing obtained by treating Hepg2.2.15 cells with four different CSLN/siHBV complexes comprising different four siRNAs (si257, si1658, si1826 and si2422) targeting four different overlapping regions of open reading frame of HBV virus genome at 0-200 nM. From the results, it was confirmed that after treated with 200 nM of CSLN/si1658 complex, HBsAG was inhibited to maximum 32.5±7.5%. This result shows that a CSLN/siRNA complex very effectively transports siRNA in the cells and the siRNA transferred in the cells induces effective inhibition of expression of target genes in the cells.

### EXAMPLE 8

### Anti-Fibrotic Effect of CSLN/siCTGF Complex

Total 36 SD male rats (Orientbio) of average 3-week old and weight of 35-40 g were divided into four groups and tested.

Control (n=6): A saline solution (0.15 M NaCl) was intraperitoneally administered daily for 7 days, and 2 hours after the intraperitoneal administration, a saline solution was intravenously administered again.

NDMA+siCTGF group (n=12): 10 mg/kg of N-nitrosodimethylamine (NDMA) was intraperitoneally administered daily for 7 days, and after 2 hours, a CSLN/siCTGF complex containing CTGF (connective tissue growth factor) siRNA (hereinafter, siCTGF; Connective tissue growth factor (CTGF) siRNA; sense: 5′-CAA UAC CUU CUG CAG GCU GGA dTdT-3′; antisense: 5′-UCC AGC CUG CAG AAG GUA UUG dTdT-3′) was intravenously administered at 1 mg/kg.

NDMA+scCTGF group (n=12): 10 mg/kg of NDMA NDMA was intraperitoneally administered daily for 7 days, and after 2 hours, a CSLN/siCTGF complex containing scrambled CTGF siRNA (hereinafter, scCTGF; sense: 5′-GGG ACG CAC UAC CUA GAC UUUtt-3′; antisense: 3′-ttCCC UGC GUG AUG GAU CUG AAA-5′)) was intravenously administered at 1 mg/kg.

NDMA+SCLN group (n=6): 10 mg/kg of NDMA was intraperitoneally administered daily for 7 days, and after 2 hours, 30 mg/kg of CSLN (Example 1) was intravenously administered.

All the administration was conducted without anesthesia, and the test animals were anesthetized with 1 mg/kg of xylazine and 50 mg/kg of ketamine at 13 days from the first administration date. And then, a blood sample was gathered, and cardiac perfusion was conducted to sacrifice the animals, and then, each organ was sampled.

The blood sample gathered from heart was centrifuged at 300 ×g for 15 minutes to separate blood serum. From the separated blood serum, the levels of alanine transaminase (ALT), aspartate transaminase (AST), total bilirubin (TBIL), and total albumin (ALB) were measured with a Fuji DRI-CHEM 3000 automatic chemistry analyzer.

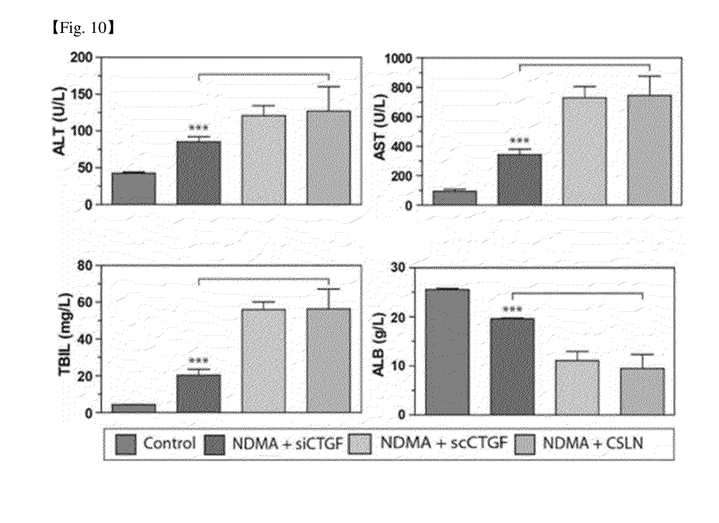

The obtained results are shown in FIG. 10. FIG. 10 shows blood ALT, AST, TBIL, ALB levels obtained by analyzing the blood sample obtained from the three different test groups and control, and it is confirmed that by treating NDMA-induced hepatic fibrosis animal models with CSLN/siCTGF complex, ALT, AST, TBIL increase and ALB decrease can be significantly inhibited.

Using the blood sample, the levels of TNF-α, TGF-β, and IL-6 included in each blood serum sample was measured with an ELISA kit (R&D Diagnostics). Using each extracted liver sample, the concentrations of CTGF and α-Tubulin included in the tissue were confirmed through western blotting.

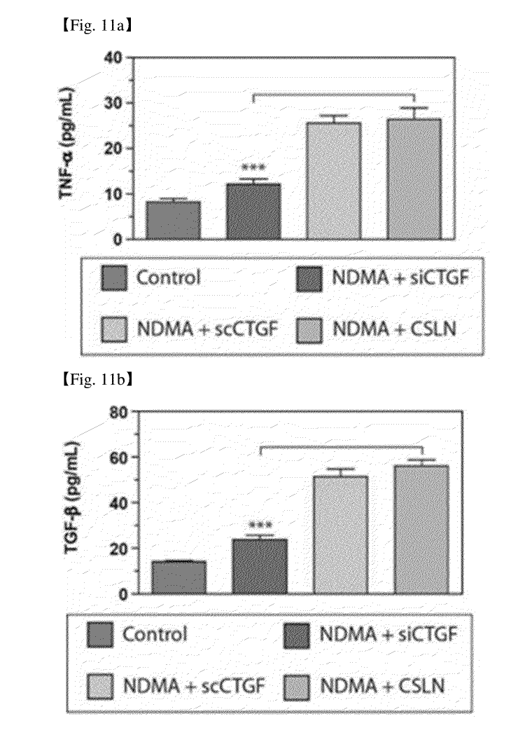

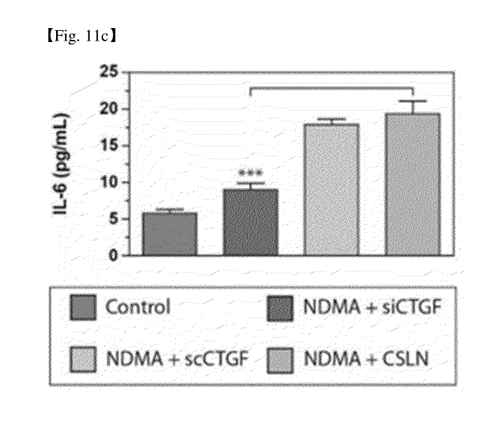

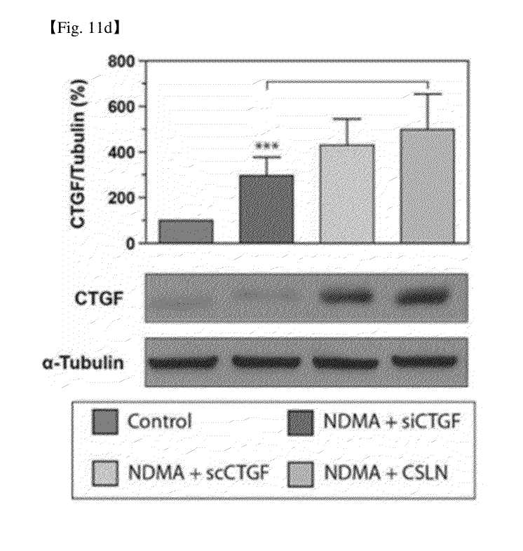

The obtained results are shown in FIG. 11a to FIG. 11d. FIG. 11a to FIG. 11d show blood TNF-α (FIG. 11a), TGF-β (FIG. 11b), and IL-6 (FIG. 11c) levels obtained by analyzing the blood sample obtained from the three different test groups and control, and the expression degrees of CTGF and tubulin in tissue analyzed using liver tissue (FIG. 11d). These results show that by treating NDMA-induced hepatic fibrosis animal models with CSLN/siCTGF complex, significant inhibition of TNF-α, TGF-β, IL-6 increase, namely anti-inflammatory and anti-fibrotic effects can be obtained, and that such effects result from inhibition of CTGF expression mediated by CSLN.

### EXAMPLE 9

### Immunofluorescent Staining

The tissue extracted in Example 8 was fixed with a 10% formalin solution, and then, paraffin-embedded. The obtained paraffin block was cut to a thickness of 5 mm using a microtome (Thermo), deparaffinized and hydrated. Specifically, the slide was put in a rack and dried at 65° C. for 1 hour. The slide was sequentially soaked in 4 containers containing xylene for each 10 minutes to remove paraffin. The paraffin-removed slide was soaked in containers containing 100, 95, 80, 70% (v/v) of ethanol for each 5 minutes, and then, washed with tap water for 5 minutes to remove ethanol and hydrate.

The obtained serial sections were respectively H&E stained and Masson's trichrome stained by commonly used standard protocol. The obtained slides were observed with brightfield microscope (Eclipse E800, Nikon) and photographed.

And, the obtained sections were treated with 1% (w/v) bovine serum albumin (BSA) containing 0.1% (v/v) Triton X-100 for 30 minutes. Thereafter, they were treated with normal goat serum (Gibco, Invitrogen, Carlsbad, Calif.) containing 0.1% (v/v) Triton X-100 for 1 hour to block. And then, they were treated with mouse anti-α-SMA antibody (1/200; Abcam, Cambridge, Mass.) and mouse anti-α-SMA antibody (1/500; Abcam (Cambridge, Mass.) and allowed to stand overnight at 4° C. After the treatment was completed, the slides were washed with PBS, and treated with Alexa flour 568 goat anti-mouse IgG (1/500; Molecular Probes, Eugene, Oreg.) or Alexa flour 488 goat anti-rabbit IgG (1/200; Molecular Probes, Eugene, Oreg.) diluted in a blocking solution (Gibco, Invitrogen, Carlsbad, Calif.) for 1 hour. The obtained slides were observed under a confocal microscope (Bio-Rad, Hercules).

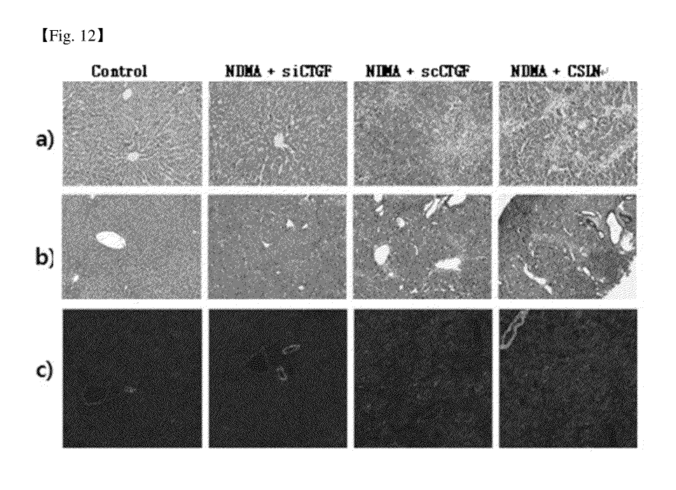

The obtained results are shown in FIG. 12. FIG. 12 shows the results of histologic and immunologic analysis of fibrotic liver tissue extracted from the animals treated with control, NDMA+CSLN/siCTGF, NDMA+CSLN/scCTGF, and NDMA+CSLN described in Example 8, wherein (a) shows the results of H&E staining of the samples obtained from each group, (b) shows the results of Masson's trichrome staining of the samples obtained from each group, and (c) shows the results of α-SMA (green), CTGF (red), and nucleus (blue) staining of the samples obtained from each group through immunofluorescent staining.

In FIG. 12 (a), in control, normal central veins and radiating hepatic cords are seen together with a normal lobular architecture and a normal hepatic cell structure, while in CSLN-treated groups, fibrosis and initial stage cirrhosis, multifocal hepatocyte necrosis, and neutrophil infiltration are observed. In scCTGF-treated group, distinct improvement effect is not seen, and extensive bridging and well developed fiber, serious centriobular congestion, sinusoid dilatation and focal hemorrhage are observed. However, in siCTGF-treated group, only mild sinusoid dilatation and neutrophil infiltration are observed, and thus, it is confirmed that SCLN-mediated CTGF siRNA treatment exhibits excellent anti-fibrotic effect.

Hepatic fibrosis is characterized by accumulation and overexpression of collagen, and finally, leads to failure.

As shown in FIG. 12 (b), any change or collagen accumulation was not observed by Masson's staining in control, while massive bridging and accumulation of mature collagen fiber were observed in the CSLN-treated group. The scCTGF-treated group did not exhibit significant difference compared to the CSLN-treated group. However, in the siCTGF-treated group, unlike other groups, only mild collagen accumulation was observed. These results accord with the H&E results showing the CSLN-mediated anti-fibrotic effect of CTGF.

In the immunohistochemical data of FIG. 12 (c), remarkable CTGF and a-SMA staining was observed in the CSLN- and scCTGF-treated groups, while only focal marginal staining was confirmed in the siCTGF-treated group.

### EXAMPLE 10

### Hepatocellular Uptake

In order to show hepatocellular uptake of CSLN/siRNA complex, a SCLN/FAM-siRNA complex (prepared by the method of Example 2) containing 1.0 mg/kg of FAM-siRNA (identical to Example 6; Invitrogen, Carlsbad, Calif.) was intravenously administered to SD male rat (average 3-week old and weight 35-40 g). 12 hours after the administration, the animal was anesthetized and perfused with PBS containing 4% formalin. The tissue was extracted and additionally fixed with formalin, followed by cryoprotection with a 30% sucrose solution. Thereafter, the tissue sample was freezed on tissue frozen medium, and then, sectioned to a thickness of 10 um with a cryotom apparatus (Thermo Co. Ltd.) and observed under a confocal microscope.

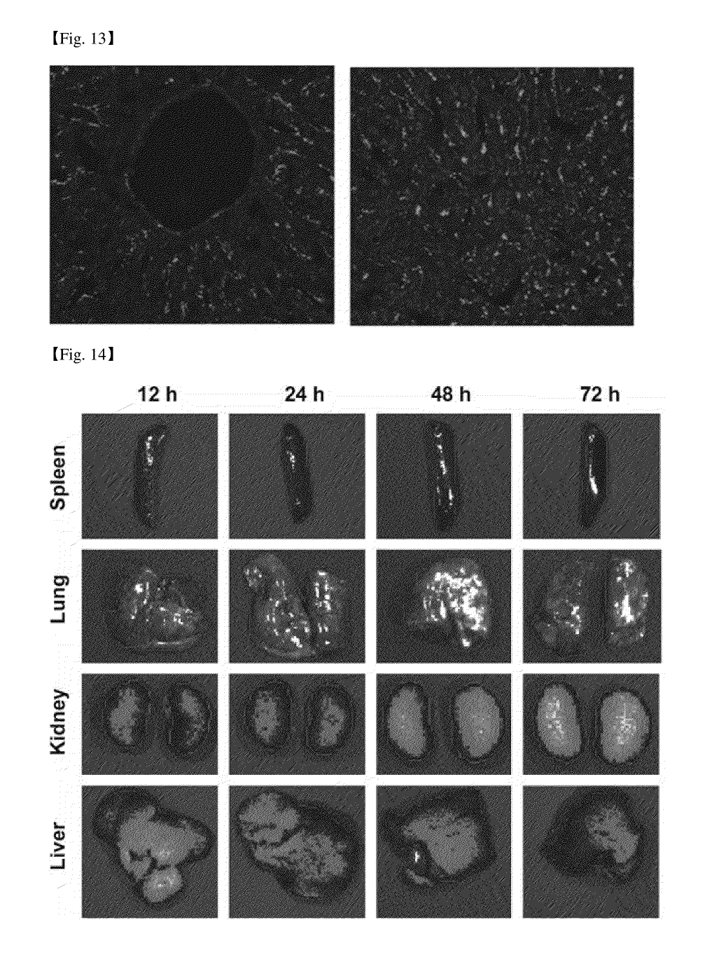

The obtained results are shown in FIG. 13. FIG. 13 shows the results of observation by cryosection of left lateral liver robe and right lateral liver robe after intravenous administration of CSLN/FAM-siRNA complex (left: left lateral liver robe, right: right lateral liver robe), and it shows that there is noticeable cellular uptake of intravenously administered CSLN/siRNA in liver parenchyma. From these results, it can be seen that LDL-like CSLN effectively delivered siRNA in liver cells, thereby exhibiting the above-mentioned treatment effect.

### EXAMPLE 11

### Quantum Dot-Loaded CSLN

In the above described CSLN preparation process, 0.5 nmole of hydrophobic Q.Dot 705 (Life Technologies (Grand Island, N.Y.)) was added to the constituents of CSLN to prepare CSLN containing quantum dot by the same method as described in Example 1. The prepared Q.Dot-containing CSLN (hereinafter, Q.Dot-CSLN) was mixed with 1 mg/kg of siRNA (siCTGF of Example 8) to form a complex at a weight ratio (Q.Dot-CSLN/siRNA) of 30 by the method of Example 2. The obtained Q.Dot-CSLN/siRNA complex was intravenously injected into 12 SD male rats of average 3-week old and weight 35-40 g, and then, the rats were sacrificed at different time points (12, 24, 48, 72 hours) and the organs were extracted, and then, fluorescence intensity remaining in each organ was analyzed with in vivo fluorescence imaging system (IVIS; Xenogen, Alameda, Calif.).

The obtained results are shown in FIG. 14. FIG. 14 shows the results of confirming by optical imaging of biodistribution of the CSNL-Q.Dot/siRNA complex over time after intravenous administration of the above obtained CSNL-Q.Dot in animals, and it shows that encapsulation of hydrophobically surface modified Quantum dot in the solid lipid core of CSLN can be simply and effectively achieved and that biodistribution of the administered SCLN/siRNA complex is liver specific.

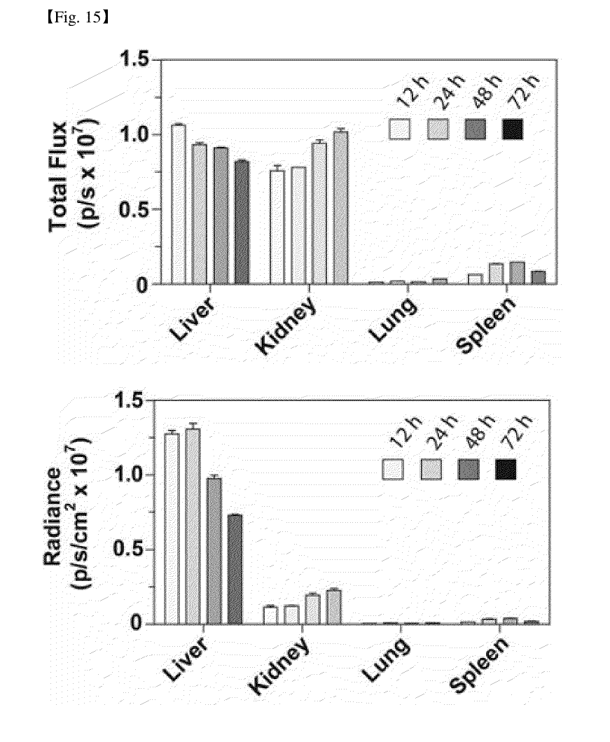

FIG. 15 shows the results of confirming distribution of CSLN/siRNA according to organs over time by software analysis of the results of FIG. 14, wherein fluorescent signal gradually increases in kidney over time after accumulation of CSLN/siRNA in liver cells, which suggests clearance of Q-Dot through kidney after LDL-mimicking metabolism of CSLN.

From these results, it was confirmed that CSLN can load a therapeutic composition comprising nucleic acid genes in the cationic outer shell, and simultaneously load an imaging composition for diagnosis such as nanoparticles such as the above described hydrophobically surface modified Q.dot and a hydrophobic therapeutic composition in the core, and liver-specifically deliver them.

### EXAMPLE 12

### Confirmation of biodistribution using Radioisotope

Among the constituents of CSLN described in Table 2 of Example 1, a part of cholesterol (0.5 μmole) was replaced with radioisotope-labeled cholesterol (Carbone-13) (Sigma-Aldrich, St. Louis, Mo.) to prepare CSLN by the same method as described in Example 1. The obtained CSLN (hereinafter, radioisotope-labeled-CSLN) was mixed with 1 mg/kg of siRNA to form a complex at a weight ratio of 30 (See Example 2). The obtained radioisotope-labeled-CSLN/siRNA complex was intravenously administered to SD male rats of average 3-week old and weight 35-40 g at a dose of 30 ug/kg based on the amount of CSLN, and after 48 hours, the animals were anesthetized to image through Siemens dual modality SPECT/MicroCAT II scanner (Knoxville, Tenn.).

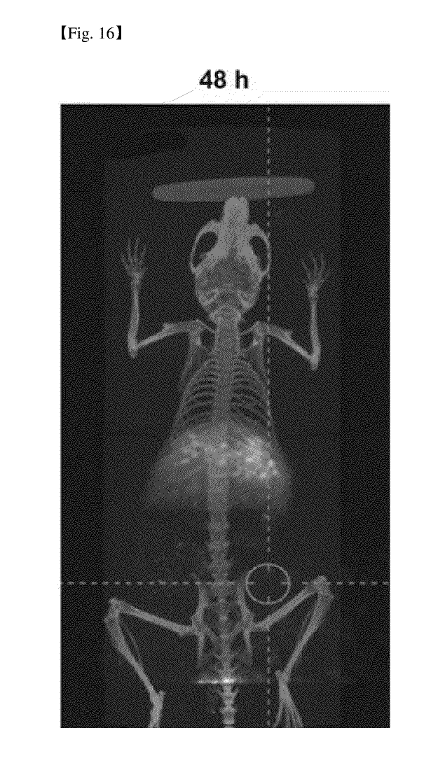

The obtained results are shown in FIG. 16. FIG. 16 shows the results of non-invasive X-ray liver imaging due to in vivo behavior of CSLN/siRNA and liver specific delivery and accumulation of CSLN, by replacing the existing lipid composition constituting CSLN with a radioisotope-labeled lipid composition. It shows that in the preparation process of CSLN, a part of the composition can be changed to simultaneously afford diagnostic and therapeutic functions, similarly to the example using Q.Dot.

1. A low density lipoprotein (LDL)-like nanoparticle with a core-shell structure comprising
a core containing cholesteryl ester and triglyceride; and
a shell containing cholesterol, fusogenic lipids, cationic lipids, and lipid-PEG (polyethyleneglycol) conjugate.

2. The LDL-like nanoparticle according to claim 1, wherein the mole ratio of the lipid and the PEG in the lipid-PEG conjugate is 1:0.5 to 3 (lipid moles:PEG moles).

3. The LDL-like nanoparticle according to claim 2, wherein the PEG in the lipid-PGE conjugate has a weight average molecular weight of 100 to 100,000 Daltons.

4. The LDL-like nanoparticle according to claim 1, wherein the lipid-PEG conjugate is distearoylphosphatidylethanolamine (DSPE)-PEG or dipalmitoylphosphatidylethanolamine (DPPE)-PEG.

5. The LDL-like nanoparticle according to claim 1, wherein the cholesterol ester is a ester compound of cholesterol to a saturated or unsaturated fatty acid having a carbon number of 10 to 24, and the triglyceride is triolein.

6. The LDL-like nanoparticle according to claim 1, wherein the fusogenic lipid is at least one selected from the group consisting of
dioleoylphosphatidylethanolamine (DOPE), palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), distearoylphosphatidylethanolamine (DSPE), phosphatidylethanolamine (PE), dipalmitoylphosphatidylethanolamine, 1 ,2-dioleyl-sn-glycero-3-phosphoethanolamine, 1-palmitoyl-2-oleyl-sn-glycero-3-phophoethanolamine (POPE), 1-palmitoyl-2-oleyl-sn-glycero-3-phosphocholine (POPC), 1 ,2-dioleyl-sn-glycero-3-[phospho-L-serine] (DOPS), and 1,2-dioleyl-sn-glycero-3-[phospho-L-serine], and
the cationic lipid is at least one selected from the group consisting of
3beta-[N—(N′,N′,N′-trimethylaminoethane)carbomoyl]cholesterol (TC-cholesterol), 3beta[N—(N′,N′-dimethylaminoethane)carbamoyl]cholesterol (DC-cholesterol), 3beta[N—(N′-monomethylaminoethane)carbamoyl]cholesterol (MC-cholesterol), 3beta[N-(aminoethane)carbamoyl]cholesterol (AC-cholesterol), N—(N′-aminoethane)carbamoylpropanoic tocopherol (AC-tocopherol), N—(N′- [symbol] aminoethane)carbamoylpropanoic tocopherol (MC-tocopherol), N,N-dioleyl-N,N-dimethylammoniumchloride (DODAC), N,N-distearyl-N,N-dimethylammoniumbromide (DDAB), N-(1-(2,3-dioleoyloxy)propyl-N,N,N-trimethylammoniumchloride (DOTAP), N,N-dimethyl-(2,3-dioleoyloxy)propylamine (DODMA), N-(1-(2,3-dioleyl)propyl)-N,N,N-trimethylammonium chloride (DOTMA), 1,2-dioleyl-3-dimethylammonium-propane (DODAP), 1 ,2-dioleylcarbamyl-3-dimethylammonium-propane (DOCDAP), 1 ,2-dilineoyl-3-dimethylammonium-propane (DLINDAP), dioleoyloxy-N-[2-sperminecarboxamido)ethyl}-N,N-dimethyl-1-propanaminium trifluoroacetate (DOSPA), dioctadecylamidoglycyl spermine (DOGS), 1,2-dimyristyloxypropyl-3-dimethyl-hydroxyethyl ammonium bromide (DMRIE), 3-dimethylamino-2-(cholest-5-en-3-beta-oxybutane-4-oxy)-1-(cis,cis-9,12-octadecadienoxy)propane (CLinDMA), 2-[5′-(cholest-5-en-3-beta-oxy)-3′-oxapentoxy]-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-diacyl-3-trimethylammonium-propane (TAP), 1,2-diacyl-3-dimethylammonium-propane (DAP), 1,2-di-O-octadecyl-3-trimethylammonium propane, and 1,2-dioleyl-3-trimethylammonium propane.

7. The LDL-like nanoparticle according to claim 1, comprising 30 to 60 wt % of cholesteryl ester, 0.1 to 10 wt % of triglyceride, 5 to 20 wt % of cholesterol, 5 to 30 wt % of fusogenic lipids, 10 to 50 wt % of cationic lipids, and 0.01 to 1 wt % of lipid-PEG conjugate, based on the total weight of nanoparticle.

8. The LDL-like nanoparticle according to claim 1, wherein the weight ratio of the core and the shell is 30:70 to 70:30.

9. The LDL-like nanoparticle according to claim 1, further comprising detection particles formed by hydrophobic surface modification of at least one nanoparticles selected from the group consisting of quantum dot, carbone dot, gold nanoparticles, and iron oxide nanoparticles in an amount of 1% (w/w) to 20% (w/w), based on the weight of the LDL-like nanoparticle.

10. The LDL-like nanoparticle according to claim 1, wherein further comprising radioisotope labeled lipids obtained by radioisotope labeling of at least one material selected from the group consisting of cholesterol, fusogenic lipid, cationic lipid and lipid-PEG conjugate in an amount of 0.001% (w/w) to 5% (w/w), based on the total weight of LDL-like nanoparticle.

11. A nanoparticle complex comprising the low density lipoprotein-like nanoparticle according to claim 1 and an active ingredient, wherein the active ingredient is nucleic acid, anionic drug, or hydrophobic drug.

12. The nanoparticle complex of an active ingredient and nanoparticle according to claim 11, wherein the nucleic acid is bonded by electrostatic interaction with the cationic lipid included in the shell of the LDL-like nanoparticle, and the weight ratio of the LDL-like nanoparticle to the nucleic acid (LDL-like nanoparticle weight/nucleic acid weight) is 10 to 50.

13. The nanoparticle complex of an active ingredient and nanoparticle according to claim 11, wherein the nucleic acid is at least one selected from the group consisting of small interfering RNA (siRNA), ribosomal ribonucleic acid (RNA), deoxyribonucleic acid (DNA), complementary DNA (cDNA), aptamer, messenger ribonucleic acid (mRNA), transfer ribonucleic acid (tRNA), and antisense oligodeoxynucleotide (AS-ODN),
the anionic drug is negatively charged peptide, protein, hyaluronic acid-peptide conjugate, or hyaluronic acid-protein conjugate, and the hydrophobic drug is taxol, doxorubicin, or epirubicin.

14. A method of liver cell specific targeting of an active ingredient using a nanoparticle complex comprising the LDL-like nanoparticle according to claim 1 and an active ingredient.

15. The method according to claim 14, wherein the LDL-like nanoparticle further comprise detection particles formed by hydrophobic surface modification of at least one nanoparticles selected from the group consisting of quantum dot, carbone dot, gold nanoparticles, and iron oxide nanoparticles in an amount of 1% (w/w) to 20% (w/w), based on the total weight of the LDL-like nanoparticles, and the detection particles are used to image the LDL-like nanoparticle.

16. The method according to claim 14, wherein the LDL-like nanoparticle further comprise radioisotope labeled lipids of at least one selected from the group consisting of cholesterol, fusogenic lipid, cationic lipid and lipid-PEG conjugate in an amount of 0.001% (w/w) to 5% (w/w), based on the total weight of LDL-like nanoparticles, and the radioisotope labeled lipids are used to detect the LDL-like nanoparticle.

17. (canceled)


SEQUENCE LISTING (6 sequences)

SEQ ID NO: 1 - DNA, 25 bp, Artificial Sequence
  siCTGF sense strand
caauaccuucugcaggcuggadtdt

SEQ ID NO: 2 - DNA, 25 bp, Artificial Sequence
  siCTGF antisense strand
uccagccugcagaagguauugdtdt

SEQ ID NO: 3 - DNA, 21 bp, Artificial Sequence
  si257 siRNA sense strand
gugguggacuucucucaauuu

SEQ ID NO: 4 - DNA, 22 bp, Artificial Sequence
  si658 siRNA sense strand
aagaggacucuuggacucucuu

SEQ ID NO: 5 - DNA, 22 bp, Artificial Sequence
  si1826 siRNA sense strand
uucaccucugccuaaucauuua

SEQ ID NO: 6 - DNA, 21 bp, Artificial Sequence
  si2422 siRNA sense strand
aagaucucaaucucgggaauc